(19) United States
(12) Patent Application Publication
(10) Pub. No.: US 2021/0038421 A1
(43) Pub. Date: Feb. 11, 2021

(54) STABILIZING SACPULAR REHABILITATION BRACE

(71) Applicant: Eduardo Marti, Weston, FL (US)

(72) Inventor: Eduardo Marti, Weston, FL (US)

(21) Appl. No.: 16/537,558

(22) Filed: Aug. 10, 2019

(51) Int. Cl. A61F 5/37; A61F 5/058

(52) U.S. Cl. CPC A61F 5/3723; A61F 5/05858

(57) ABSTRACT

A stabilizing scapular rehabilitation brace that operates to aid patients in rehabilitating their shoulder joint in an anatomically correct manner with the trapezius is locked down in order to prevent substantial upward shoulder shrug movements. The stabilizing scapular rehabilitation brace includes a shoulder harness, a stabilization strap, and a thigh strap, with the stabilization strap operating to both tighten the shoulder harness on a user and connect and anchor the shoulder harness to the thigh strap. With the shoulder harness operating to keep the user's shoulder blades retracted and the thigh strap and stabilization strap preventing the shoulder harness, these components work together to emulate the same “hand on trapezius area” function that therapists manually apply to patient rehabbing a shoulder condition in-clinic.

### BACKGROUND OF THE INVENTION

### Field of the Invention

This invention relates to a stabilizing brace for use following injury to or surgery in the shoulder area that prevents a user from engaging in compensatory motion while moving their arm on during their physical therapy rehabilitation sessions.

### Description of the Prior Art

The shoulder is the joint with the greatest amount of movement in the body. The shoulder is like a ball and socket, except that the socket is shallow which provides greater movement mechanics but less stability. Instability is a common problem and is the root of many shoulder injuries for not only young athletes but individuals of all ages. Indeed, while young athletes may often suffer from acute injuries, older individuals commonly suffer from the result of years and years of use and the muscular requirements to stabilize the shoulder. In any event, whether from injuries, overuse, or cumulative effects, shoulder conditions often become the leading cause of shoulder pain.

The shoulder moves in three axis of motion. One such motion is commonly referred to as abduction and adduction. Abduct means move away from the body and adduction toward the body. Another such motion is commonly referred to as internal and external. These can be envisioned from a starting point of one's elbow down to their side and their elbow bent at a ninety degree angle with hand facing forward. Moving the hand toward the center of your body with the elbow still at your side is internal rotation. Moving the hand away with elbow still at your side is external motion. Further, moving the elbow out to the side and while still moving the hand back and forth with it at your side is internal and external rotation in abduction. Yet another such motion is commonly referred to as transverse (or horizontal) rotation. This motion describes the act of going around the body center in a circular motion.

The shoulder girdle encompasses the junction between four major bones of the upper torso, namely, the sternum (breastbone), clavicle (collarbone), the scapula (shoulder blade), and the humerus (upper arm). The clavicle via the sternum serves as the bony attachment between the shoulder and the trunk itself. The other two bones of the shoulder are attached to the trunk by muscles and ligaments alone.

The outer end of the clavicle is attached to the scapula by the non-axial (i.e., limited to gliding movements) acromio-clavicular joint (AC-joint). The humerus is attached to the scapula by the tri-axial (i.e., ability to move in three planes) glenohumeral joint. This joint is composed of the humeral head and the “socket” (glenoid fossa) of the scapula. The unique articulative relationship of these two joints and the third joint of the shoulder girdle, the non-axial sterno-clavicular joint (located between the clavicle and the sternum) allows the shoulder to have an incredible range of motion.

The scapula, which is commonly referred to as the shoulder blade, is the triangular shaped bone on each side of a person's upper back. The socket of the shoulder joint is a part of the scapula. There are only three muscles that are responsible for enabling the movement of the shoulder blade. The first is the trapezius muscle, which implants into the collarbone and is responsible for movement of the shoulder and head. The second is the levator muscle, which is a small, thin muscle that arises from the vertebrae of the neck. A small tendon attaches the levator to the upper area of the shoulder blade. The levator is responsible for pulling up the scapula, which allows for the shrugging movement of the shoulders. The third is the rhomboideus, which actually comprises two muscles, the major and minor, located deep in the base of the shoulder blade. These rhomboideus muscles are responsible for raising the shoulder blade and moving it backwards. Separately, the muscles that move the shoulder forward come from the breast and upward movements are controlled by muscles located in the neck.

Normally the scapula will slide flat on the ribcage and rotate normally when an individual brings arms overhead. The scapula helps keep the shoulder centered in its socket and minimizes stress on the subacromial space. The scapula is not supposed to be the primary form of movement when the patient is engaged in shoulder movement that is not overhead.

Following shoulder injury or surgery in the shoulder area, however, it is often difficult and/or painful for an individual to properly move/use their arm/shoulder. As a result, the body compensates, and patients may rely too much on their scapula to help move their arm during rehabilitation (or “rehab”). This compensatory movement is called “shoulder hiking.” Shoulder hiking can present many problems because following shoulder injury or surgery, it is imperative that the shoulder joint and/or shoulder capsule be rehabilitated properly to regain full range of motion and strength. If shoulder hiking takes place, it means the patient is essentially “cheating” their rehab efforts because in moving their arm, they are creating shoulder movement with the scapula rather than the shoulder joint. But since the shoulder joint should be driving all shoulder/arm movement that is not overhead, when patient relies on scapula to be the primary form of moving the shoulder (below head level), it will produce poor outcomes that will have short and long term consequences, both physical and financial.

Common problems that may require the use of measures which will prevent shoulder hiking during rehab include, but are not limited to: rotator cuff tears and tendinitis; shoulder bursitis; shoulder surgery; frozen shoulder; shoulder dislocation; after upper extremity fracture; and scapular dyskinesia.

There exist many types of shoulder orthotics in the marketplace, and they all have essentially one underlying theme in common: they provide the support and stability your shoulder needs to heal after an injury or surgery. Such braces are typically designed to limit motion, which helps reduce pain from exaggerated movement following injury or surgery. Also, by keeping the shoulder joint in a secure position these braces help protect against potential re-injury to the actual surgical repair performed on the shoulder joint.

Shoulder stabilization braces (or shoulder immobilization braces) are one type of shoulder orthotic. Shoulder stabilization braces are designed to provide shoulder immobilization and controlled range of motion for glenohumeral dislocations/subluxations, rotator cuff tears and acromioclavicular separations following surgery. They are designed to protect and stabilize the shoulder post-injury and post-operatively. They are not, however, suitable or intended to be used during rehab sessions.

Shoulder slings are another type of shoulder orthotic used to support arm after injury or surgery. Shoulder slings may also be used to limit movement to reduce pain and swelling. They are not, however, suitable for use as a rehab brace.

Another type of shoulder orthotic, posture braces (or posture correctors), function by limiting forward shoulder movements and restricting slouching and bending of the spine. Posture braces provide gentle yet firm support that results in a straighter, taller spine. They are not, however designed to and are generally unable to prevent shoulder hiking during rehab movements when patients begin rehabilitation following shoulder injury or surgery. And this is a major problem because limiting a patient's compensational shoulder hiking movement is critical in order to achieve optimal outcomes.

While all of these types of shoulder orthotics play an important role in patient healing, it is clear that they are not rehab braces that can prevent shoulder hiking during rehab activities. Accordingly, there remains a need for a stabilizing scapular rehabilitation brace which can aid patients during their rehab exercises by promoting proper trapezoid and scapular positioning and preventing shoulder hiking during rehabilitative sessions.

### SUMMARY OF THE INVENTION

The present disclosure provides for a stabilizing scapular rehabilitation brace, comprising: a shoulder harness having at least one shoulder strap and at least one strap connector disposed on the at least one shoulder strap, wherein the shoulder harness is configured to be worn by a user with the at least one shoulder strap going over their shoulders; a thigh strap configured to be fastened around a user's thigh; and a stabilization strap having a top end and a bottom end, wherein the stabilization strap includes a harness connector that is adapted to be removably attached to the at least one strap connector integral with the top end and the stabilization strap is adapted to be coupled with the thigh strap at the bottom end.

### BRIEF DESCRIPTION OF THE DRAWINGS

FIG. 1 is a front perspective view of a scapular stabilization technique for rehabilitation activities in accordance with the prior art.

FIG. 2 is a front perspective view of a stabilizing scapular rehabilitation brace built in accordance with an embodiment of the present invention.

FIG. 3 is a front perspective view of a shoulder harness for a stabilizing scapular rehabilitation brace built in accordance with an embodiment of the present invention.

FIG. 4 is a front perspective view of a stabilization strap for a stabilizing scapular rehabilitation brace built in accordance with an embodiment of the present invention.

FIG. 5 is a front perspective view of a thigh strap for a stabilizing scapular rehabilitation brace built in accordance with an embodiment of the present invention.

FIG. 6 is a partial side perspective view of a stabilizing scapular rehabilitation brace built in accordance with an embodiment of the present invention, shown with the stabilization strap being positioned to attach to the shoulder harness.

FIG. 7 is a partial side perspective view of a stabilizing scapular rehabilitation brace built in accordance with an embodiment of the present invention, shown with the stabilization strap being attached to the shoulder harness.

FIG. 8 is a partial front elevational view of a stabilization strap for a stabilizing scapular rehabilitation brace built in accordance with an embodiment of the present invention, shown with its tension being adjusted.

FIG. 9 is a partial side perspective view of a stabilizing scapular rehabilitation brace built in accordance with an embodiment of the present invention, shown with the stabilization strap being positioned to attach to the thigh harness.

FIG. 10 is a partial side perspective view of a stabilizing scapular rehabilitation brace built in accordance with an embodiment of the present invention, shown with the stabilization strap attached to the thigh harness.

### DETAILED DESCRIPTION OF THE INVENTION

Described herein is a stabilizing scapular rehabilitation brace that operates to aid patients in rehabilitating their shoulder joint in an anatomically correct manner to optimize outcomes following injury or surgery so as to deliver vastly improved convenience and benefits relative to existing options in the marketplace. When patients engage in shoulder hiking movements during rehabilitation (or rehab) sessions, it negatively impacts their ability to regain proper shoulder joint functionality and strength of shoulder following injury or surgery. But when the stabilizing scapular rehabilitation brace is worn during shoulder rehab exercises, forces the patient to perform exercises in an anatomically correct manner that will optimize patient's recovery. The stabilizing scapular rehabilitation brace is additionally adjustable so that it can correctly fit on all body types.

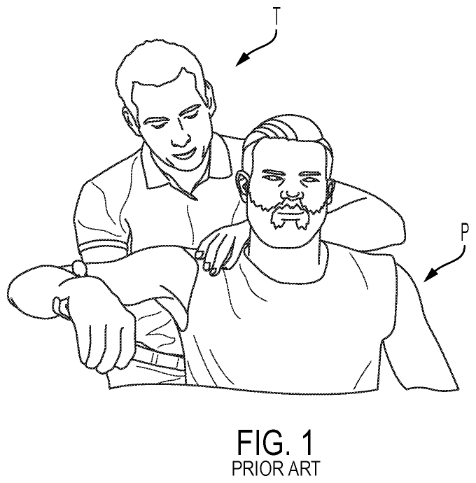

Referring now to the drawings and, in particular, FIG. 1, a common existing technique to reduce shoulder hiking involves a physical therapist T placing their hand on the patient's P trapezius area during in-clinic shoulder rehab sessions. When the patient is at home and trying to do the adjunct home-based exercises prescribed by the therapist, however, there is often no one there to manually “hold down” the trapezius area in order to prevent patient from engaging in the act of shoulder hiking. This unfortunately results in many patients, despite their best efforts, failing to regain full range of motion and strength and even possibly requiring additional surgery to resolve their shoulder problems due to their inability to rehab properly.

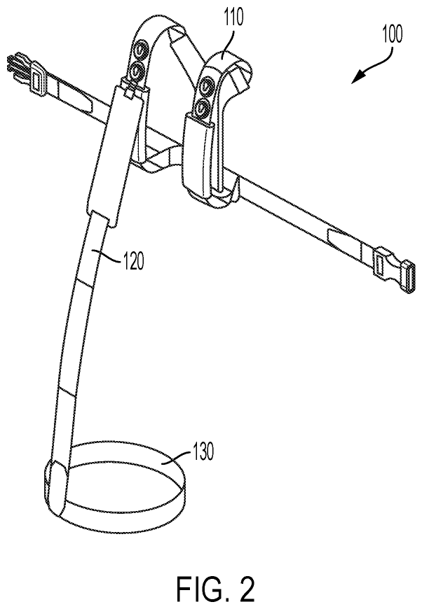

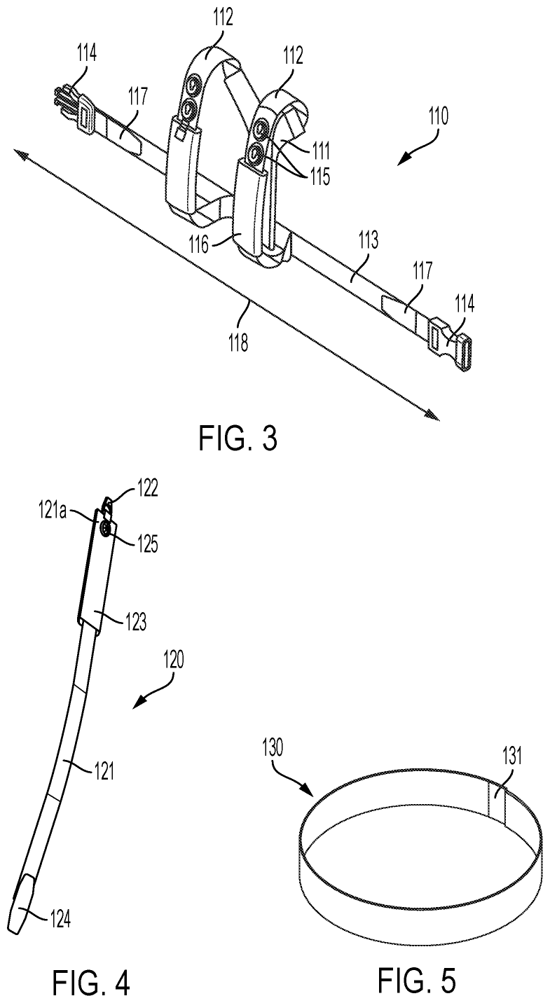

Referring now to FIGS. 2, 3, 4, and 5, a stabilizing scapular rehabilitation brace 100 is shown having a shoulder harness 110, a stabilization strap 120, and a thigh strap 130. When worn by a user, these components work together to emulate the same “hand on trapezius area” function that a therapist manually applies to patient in-clinic. This means users can rehab at any time and do so in an anatomically correct manner as the trapezius is locked down in order to prevent substantial upward shoulder shrug movements (i.e., shoulder hiking) as well as to promote retraction of the shoulder blades in the mid-back area of the trapezius.

The shoulder harness 110 includes of a padded back panel 111, shoulder straps 112, a chest strap 113, and a waist buckle component 114 having two parts that are mechanically attached (by sewing or otherwise) to the opposite ends of the chest strap 113. The shoulder harness 110 additionally includes strap connectors in the form of two quick release female connectors 115 positioned on each shoulder strap 112, as well as a protective pad 116 below the female connectors 115 on each of the shoulder straps 112 to prevent shoulder straps 112 from cutting into the skin or causing discomfort. To facilitate adjustments to the circumference of the shoulder harness 110 and accommodate larger patients by increasing length of strapping system, a length adjustment mechanism 117 may be integrated into the chest strap 113.

The length adjustment mechanism 117 may be embodied as a discrete Y tab adjustor (or the hook aspect of another hook and loop fastener material). The length adjustment mechanism 117 may operate with adjustment buckles built into the waist buckle component 114, allowing the length adjustment mechanism 117 to be pulled further away from the ends of the chest strap 113, causing chest strap 113 material to pull through the adjustment buckles built into the waist buckle component 114 (reducing the length of the chest strap 113) and then removably attached to the chest strap 113 at that location.

To put on the shoulder harness 110, a user may place the shoulder harness 110 onto their upper torso (as one does when placing a backpack on themselves), with the back panel 111 against their back, the shoulder straps 112 going over their shoulders, the portion of the shoulder straps 112 containing the female connectors 115 in front of their shoulders, and the two ends of the chest strap 113 hanging from the bottom of the back panel 111. The user then pulls the ends of the chest strap 113 around their body so that the two waist buckle components 114 can connect in front of the user's body fastens the waist buckle components 114 together and adjusts the length adjustment mechanism 117. During this process, the user pulls on the chest strap 113 to tighten it as much as possible so that the shoulder harness 110 will cradle their body. Advantageously, doing this will help keep their shoulder blades in a retracted position because the tighter the shoulder harness 110 cradles the body, the more it retracts the shoulder blades.

With the chest strap 113 tightened and fastened, it causes the shoulder straps 112 to exert force on the user's shoulders to secure the shoulder blades in a retracted position. This occurs due to the counter-pressure the “X” crisscross design of the back panel 111 applies on the shoulder blades as they retract/compress toward the spine. To explain, when a user pulls outwardly 118 on the ends of the chest strap 113, the shoulder straps 112 tighten due to the fact that they are attach to one another. Particularly, the left shoulder strap 112 is tightened by pulling the right end of the chest strap 113 and the right shoulder strap 112 is tightened by pulling the left end of the chest strap 113.

Alternatively, the user may connect the ends of the chest straps 113 together via the waist buckle components 114 after proper shoulder blade retraction tension is achieved.

The stabilization strap 120 is operative to couple the shoulder harness 110 and the thigh strap 130. The stabilization strap 120 include an elongated body 121, a harness connector in the form of a quick release clip segment 122 positioned at the top end of the elongated body 121, a padded tensioner housing 123 positioned adjacent to a top end of the elongated body 121, and a fastening portion 124 which defines the bottom end of the elongated body 121. The quick release clip segment 122 is defined by a corresponding male fastener segment for the female connectors 115, and may be embodied as a stud, hook, or snap fastener style connector (with the female connectors 115 being a corresponding socket aspect thereof) that can be inserted into and releasably connect to the female connectors 115 on the shoulder harness 110. The tensioner housing 123 houses a strap length adjustment mechanism defined as a cable ratchet system and includes a tension adjustment knob 125 for adjusting the tension applied by the cable ratchet system (as discussed below). The fastening portion 124 may be defined by a Y Tab fastener (or the hook aspect of another hook and loop fastener material). The cable ratchet system is positioned between the quick release clip segment 122 and the fastening portion 124 and is used to create tension in the stabilization strap 120 which is designed to limit undesirable shoulder hiking motions.

The cable ratchet system may operate with an extension segment 121a of the elongated body 121. In some embodiments, the elongated body 121 may extend from the bottom end to the tensioner housing 123, with the extension segment 121a being the portion of the stabilization strap that extends from the tensioner housing 123 to the top end (with the quick release clip segment 122 attached to the extension segment 121a), with the cable ratchet system operative to shorten or lengthen the extension segment 121a so as to extend or retract the length of the stabilization strap 120.

In some embodiments, the elongated body 121 may extend for the full length of the stabilization strap 120, with the extension segment 121a simply being a portion of the elongated body 121. In such an embodiment, the cable ratchet system may operate to constrict or bunch up a portion of the elongated body 121 in the tensioner housing 123 to lengthen or shorten the amount of the elongated body 121 that extends out of the tensioner housing 123 as the extension segment 121a.

The quick release clip segment 122 can be attached to either female connector 115 on the shoulder harness 110. On any given shoulder strap 112, the upper female connector 115 may used when user rehabs in a seated position and the lower female connector 115 is used when user rehabs in a standing position. After stabilization strap 120 is connected to one of the female connector 115, the user may then attach the bottom end of the stabilization strap 120 the thigh strap 130 as discussed below.

In order to properly fit the user, prior to using this brace, the bottom end of the stabilization strap 120 may be cut in length to properly account for user's height. Once the extra length from the bottom end of the stabilization strap 120 is cut and properly sized for patient then the fastening portion 124 is placed at bottom end of the stabilization strap 120. Then, the fastening portion 124 is secured onto thigh strap 130. Once fitted for proper user length, the user will use the cable ratchet system to create the proper amount of tension required to eliminate shoulder hiking during their rehab sessions.

The thigh strap 130 is defined by a circular strap member that is sized to encircle and otherwise operative to be fastened around the user's upper thigh. The thigh strap 130 has a loop adjustment mechanism 131 that allows for adjustment to its circumference in order to ensure proper patient fitting. The loop adjustment mechanism 131 may be embodied as a buckle.

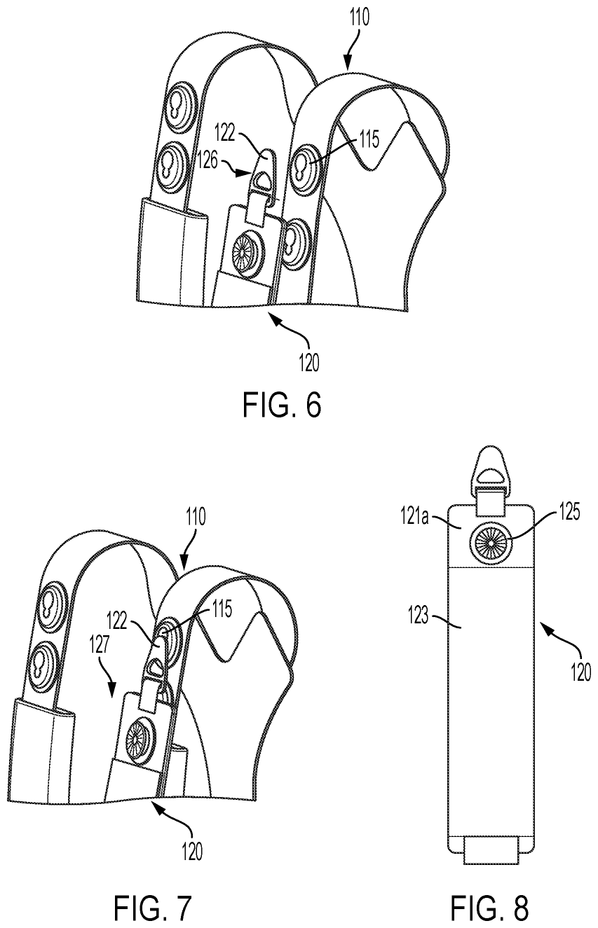

The stabilizing scapular rehabilitation brace 100 allows a user to perform exercises while seated or standing depending how the user connects the stabilization strap 120 to the shoulder harness 110. The manner in which the stabilization strap 120 is connected to the shoulder harness 110 is illustrated in FIGS. 6, 7, and 8.

Referring now to FIGS. 6 and 7, to attach the stabilization strap 120 to the shoulder harness 110, the quick release clip segment 122 must be aligned with one of the female connectors 115. As mentioned above, the upper female connector 115 is for use when the user is seated and the lower female connector 115 is for use when the user is standing. As soon as the stud from the quick release clip segment 122 from the stabilization strap 120 is pushed in a insertion direction 126 into the wide portion of the socket of the female connector 115, the user pulls in the securing direction 127 to move the stud into the narrower portion of the socket of the female connector 115. The user may hear an audible click once attachment is successful.

Referring now to FIG. 8, to adjust how much of the extension segment 121a extends from the tensioner housing 123 (and thus length of the stabilization strap 120), a user may rotate the tension adjustment knob 125 to activate the cable ratchet system and lengthens or shortens the extension segment 121a depending on the direction which the tension adjustment knob 125 is rotated.

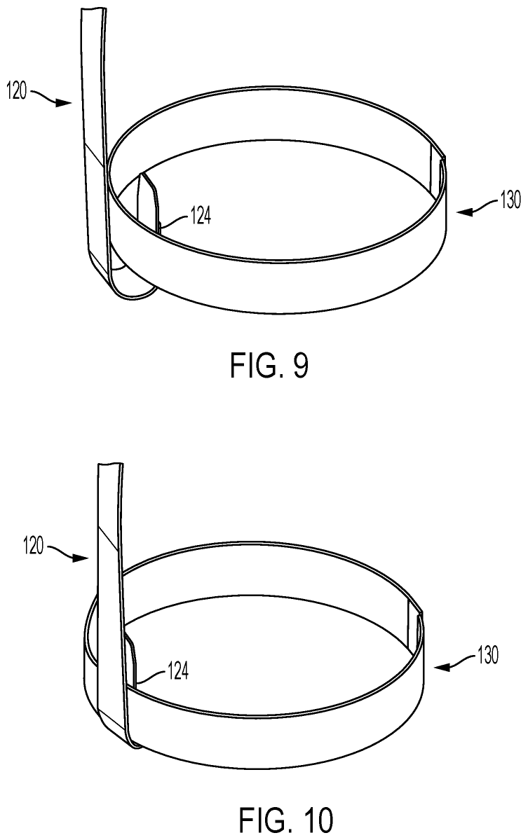

Referring now to FIGS. 9 and 10, the stabilization strap 120 may be secured onto the thigh strap 130 via the fastening portion 124 being wrapped around the thigh strap 130 and fastened to the interior side of the thigh strap 130 and/or the stabilization strap 120. When the stabilization strap 120 is connected to shoulder harness 110, then fastened onto the thigh strap 130, the stabilizing scapular rehabilitation brace 100 prevents shoulder hiking in part because the thigh strap 130 acts as an anchor which assists the shoulder harness 110 in preventing upward shoulder hiking.

When in use, the stabilizing scapular rehabilitation brace 100 helps a user regain full range of motion and strength by ensuring that the user performs their rehab exercises in an anatomical correct manner during shoulder rehab sessions. The stabilizing scapular rehabilitation brace 100 eliminates patient cheating during exercise sessions as it prevents shoulder hiking from taking place during rehab. Since cheating in the form of shoulder hiking significantly impacts user's ability to rehab correctly, the stabilizing scapular rehabilitation brace 100 reduces the likelihood of prolonged rehab sessions and reduces the likelihood of frozen shoulder and secondary surgery such as an MUA or Lysis of Adhesion Surgery due to poor rehab outcomes.

More specifically, the stabilizing scapular rehabilitation brace 100 prevents users from engaging in an upward motion with their trapezoid (e.g. shoulder shrug) by virtue of the stabilization strap which connects to the shoulder harness and thigh strap. The stabilizing scapular rehabilitation brace 100 keeps a user's shoulder blades in a retracted position, namely compressed inward towards the spine, and locks down the top of the trapezoid to prevent it from engaging in an upward motion during rehab. Such upward motion is referred to as scapular hiking or shoulder shrug. When a patient goes thru rehab without properly addressing scapular hiking issues during rehab, it permanently alters proper mechanics. Such an improper alignment leads to long term complications which will limit quality of life and arm functionality and may lead to additional therapy and/or surgery.

It is contemplated that the strap aspects of the shoulder harness, the thigh strap, and the stabilization strap may be formed of nylon with at least a portion having a surface that is analogous to a loop component of a hook and loop fastener.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A stabilizing scapular rehabilitation brace, comprising:
a shoulder harness having at least one shoulder strap and at least one strap connector disposed on the at least one shoulder strap, wherein the shoulder harness is configured to be worn by a user with the at least one shoulder strap going over their shoulders;
a thigh strap configured to be fastened around a user's thigh; and
a stabilization strap having a top end and a bottom end, wherein the stabilization strap includes a harness connector that is adapted to be removably attached to the at least one strap connector integral with the top end and the stabilization strap is adapted to be coupled with the thigh strap at the bottom end.

2. The stabilizing scapular rehabilitation brace of claim 1, wherein the shoulder harness additionally includes a chest strap and is additionally configured to be worn by a user with the chest strap encircling the user's torso.

3. The stabilizing scapular rehabilitation brace of claim 2, wherein the chest strap includes a length adjustment mechanism operative to selectively lengthen and shorten the chest strap.

4. The stabilizing scapular rehabilitation brace of claim 2, wherein the shoulder harness additionally includes a back panel connected to the chest strap and the at least one shoulder strap.

5. The stabilizing scapular rehabilitation brace of claim 1, wherein the at least one strap connector is defined by a female connector that includes a socket.

6. The stabilizing scapular rehabilitation brace of claim 5, wherein the harness connector is defined by a quick release clip segment which includes a portion that is adapted to engage with the socket.

7. The stabilizing scapular rehabilitation brace of claim 1, wherein the shoulder harness includes two discrete shoulder straps and is configured to be worn by a user with the each of the shoulder straps going a separate shoulder.

8. The stabilizing scapular rehabilitation brace of claim 7, wherein each shoulder strap includes two strap connectors, with the strap connectors on each shoulder strap arranged linearly with respect to one another, positioned on non overlapping lateral portions of the shoulder strap.

9. The stabilizing scapular rehabilitation brace of claim 1, wherein the at least one shoulder strap includes two strap connectors, with the strap connectors on the at least one shoulder strap arranged linearly with respect to one another, positioned on non overlapping lateral portions of the at least one shoulder strap.

10. The stabilizing scapular rehabilitation brace of claim 1, wherein the stabilization strap includes a strap length adjustment mechanism operative to selectively lengthen and shorten the stabilization strap.

11. A stabilizing scapular rehabilitation brace, comprising:
a shoulder harness having a chest strap, two discrete shoulder straps, at least one strap connector disposed each of the shoulder straps, and a back panel connected to the chest strap and the shoulder straps, wherein the shoulder harness is configured to be worn by a user with the each of the shoulder straps going a separate shoulder and with the chest strap encircling the user's torso;
a thigh strap configured to be fastened around a user's thigh; and
a stabilization strap having a top end and a bottom end, wherein the stabilization strap includes a harness connector that is adapted to be removably attached to the at least one strap connector integral with the top end and the stabilization strap is adapted to be coupled with the thigh strap at the bottom end.

12. The stabilizing scapular rehabilitation brace of claim 11, wherein the chest strap includes a length adjustment mechanism operative to selectively lengthen and shorten the chest strap.

13. The stabilizing scapular rehabilitation brace of claim 11, wherein the at least one strap connector is defined by a female connector that includes a socket.

14. The stabilizing scapular rehabilitation brace of claim 13, wherein the harness connector is defined by a quick release clip segment which includes a portion that is adapted to engage with the socket.

15. The stabilizing scapular rehabilitation brace of claim 11, wherein each shoulder strap includes two strap connectors, with the strap connectors on each shoulder strap arranged linearly with respect to one another, positioned on non overlapping lateral portions of the shoulder strap.

16. The stabilizing scapular rehabilitation brace of claim 11, wherein the stabilization strap includes a strap length adjustment mechanism operative to selectively lengthen and shorten the stabilization strap.

17. A stabilizing scapular rehabilitation brace, comprising:
a shoulder harness having a chest strap, two discrete shoulder straps, a plurality of strap connector disposed each of the shoulder straps, and a back panel connected to the chest strap and the shoulder straps, wherein the shoulder harness is configured to be worn by a user with the each of the shoulder straps going a separate shoulder and with the chest strap encircling the user's torso;
wherein the plurality of strap connectors on each shoulder strap are arranged linearly with respect to one another, positioned on non overlapping lateral portions of the shoulder strap;
a thigh strap configured to be fastened around a user's thigh; and
a stabilization strap having a top end and a bottom end, wherein the stabilization strap includes a harness connector that is adapted to be removably attached to any of the strap connectors integral with the top end and the stabilization strap is adapted to be coupled with the thigh strap at the bottom end.

18. The stabilizing scapular rehabilitation brace of claim 17, wherein each strap connector is defined by a female connector that includes a socket.

19. The stabilizing scapular rehabilitation brace of claim 18, wherein the harness connector is defined by a quick release clip segment which includes a portion that is adapted to engage with the socket.

20. The stabilizing scapular rehabilitation brace of claim 19, wherein:
the stabilization strap includes a strap length adjustment mechanism operative to selectively lengthen and shorten the stabilization strap; and
the chest strap includes a length adjustment mechanism operative to selectively lengthen and shorten the chest strap.
